SEQUENCE LISTING (18 sequences)

SEQ ID NO: 1 - DNA, 32 bp, ARTIFICIAL SEQUENCE
  ARTIFICIAL PRIMER FOR rrnHv
aataatctgaagcgcttggatacgttgtaggg

SEQ ID NO: 2 - DNA, 34 bp, ARTIFICIAL SEQUENCE
  ARTIFICIAL PRIMER FOR rrnHv
ccccccatggatgccataagtccctccctacaac

SEQ ID NO: 3 - DNA, 37 bp, ARTIFICIAL SEQUENCE
  ARTIFICIAL PRIMER HVRRNF
ccccctctagactcgagttttttctattttgacttac

SEQ ID NO: 4 - DNA, 33 bp, ARTIFICIAL SEQUENCE
  ARTIFICIAL PRIMER SAR5F
cccgcatgccttaggttttctagttggatttgc

SEQ ID NO: 5 - DNA, 27 bp, ARTIFICIAL SEQUENCE
  ARTIFICIAL PRIMER XR3R
ggagcccgggagttcgctcccagaaat

SEQ ID NO: 6 - DNA, 33 bp, ARTIFICIAL SEQUENCE
  ARTIFICIAL PRIMER RRT
cgcgtcccgggcgaatacgaagcgcttggatac

SEQ ID NO: 7 - DNA, 33 bp, ARTIFICIAL SEQUENCE
  ARTIFICIAL PRIMER RRB
catggatccctccctacaactgtatccaagcgc

SEQ ID NO: 8 - DNA, 32 bp, ARTIFICIAL SEQUENCE
  ARTIFICIAL PRIMER PSBA5F
cccaagcttctgcaggcctagtctataggagg

SEQ ID NO: 9 - DNA, 33 bp, ARTIFICIAL SEQUENCE
  ARTIFICIAL PRIMER PSBA5R
gggaagcttggatcctaaggaatatagctcttc

SEQ ID NO: 10 - DNA, 27 bp, ARTIFICIAL SEQUENCE
  ARTIFICIAL PRIMER BARF
cccccccatgggcccagaacgacgccc

SEQ ID NO: 11 - DNA, 24 bp, ARTIFICIAL SEQUENCE
  ARTIFICIAL PRIMER BARR
ttattagatctcggtgacgggcag

SEQ ID NO: 12 - DNA, 23 bp, ARTIFICIAL SEQUENCE
  ARTIFICIAL LINKER
ttagggcccgggaaagcggccgc

SEQ ID NO: 13 - DNA, 23 bp, ARTIFICIAL SEQUENCE
  ARTIFICIAL LINKER
taagccgccgctttcccgggccc

SEQ ID NO: 14 - DNA, 418 bp, UNKNOWN
  ARTIFICIAL rrnHv PROMOTOR
ctgcaggcctagtctataggaggttttgaaaagaaaggagcaataatcattttcttgttctatcaagagggtgctattgctcctttctttttttctttttatttatttactagtattttacttacatagacttttttgtttacattatagaaaaagaaggagaggttattttcttgcatttattcatgattgagtattctattttgattttgtatttgtttaaattgtgaaatagaacttgtttctcttcttgctaatgttactatatctttttgattttttttttccaaaaaaaaaatcaaattttgacttcttcttatctcttatctttgaatatctcttatctttgaaataataatatcattgaaataagaaagaagagctatattccttaggatccactagttctagagcggcc

SEQ ID NO: 15 - DNA, 174 bp, UNKNOWN
  ARTIFICIAL rrnBn PROMOTOR
ctcgagttttttctattttgacttactcccccgccacgagcgaacgagaatggataagaggcttgtgggattgacgtgatagggtagggttggctatactgctggtggcgaactccaggctaataatctgaagcgcttggatacgttgtagggagggacttatggcatccatgg

SEQ ID NO: 16 - DNA, 183 bp, Nicotiana tabacum
gatgaattcgatcccgcatgccttaggttttctagttggatttgctccctcgctgtgatcgaataagaatggataagaggctcgtgggattgacgtgagggggtaggggtagctatatttctgggagcgaactcccgggcgaatacgaagcgcttggatacagttgtagggagggatccatgg

SEQ ID NO: 17 - DNA, 572 bp, Streptomyces hygroscopicus
ccatgggcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctaataaatcgaattcctgcag

SEQ ID NO: 18 - protein, 183 aa, Streptomyces hygroscopicus
MGPERRPADIRRATEADMPAVCTIVNHYIETSTVNFRTEPQEPQEWTDDLVRLRERYPWLVAEVDGEVAGIAYAGPWKARNAYDWTAESTVYVSPRHQRTGLGSTLYTHLLKSLEAQGFKSVVAVIGLPNDPSVRMHEALGYAPRGMLRAAGFKHGNWHDVGFWQLDFSLPVPPRPVLPVTEI

(19) United States
(12) Patent Application Publication
(10) Pub. No.: US 2003/0188337 A1
(43) Pub. Date: Oct. 2, 2003

(54) Transgenic plants

(76) Inventors: Anil Day, Cheshire (GB); Siriluck Iamtham, Nontaburee (TH); Mikhajo Zubko, Cheshire (GB)

Correspondence Address: Joshua R Slavitt, Synnestvedt & Lechner, 1101 Market Street, 2600 Aramark Tower, Philadelphia, PA, 19107-2950, US

(21) Appl. No.: 10/258,253

(22) PCT Filed: Apr. 20, 2001
(86) PCT No.: PCT/GB01/01761

(30) Foreign Application Priority Data
Apr. 20, 2000 (GB) 0009780.8
Apr. 25, 2000 (GB) 0009968.9
Jul. 15, 2000 (GB) 0017338.5

(51) Int. Cl. A01H 1/00; C12N 15/82

(52) U.S. Cl. 800/279; 800/288

(57) ABSTRACT

The invention provides method for producing a transgenic plant comprising a recombinant plastid genome containing an exogenous gene in the absence of a selectable marker gene introduced with the exogenous gene by using direct repeat sequences, nucleic acid constructs containing direct repeat sequences which may be used in the method and transgenic plants produced by the method.

The present invention relates to transgenic plants and nucleic acid constructs and methods for the production thereof.

Modern gene transfer technologies allow the rapid development of transgenic plants with desirable properties. The scope of the technology is wide and the potential benefits to society great. For example, transgenic plants provide a means for increasing the quantity and quality of food as well as providing a renewable source of organic compounds for industry.

The escape of transgenes from crops to weedy relatives has aroused public concern about their possible deleterious effects on the environment. Further there is concern that transgenes may be able to pass from crops to humans. Methods that reduce the risk of escape of transgenes from crops are therefore of considerable benefit to the acceptance of transgenic crops in agriculture.

The majority of gene transfer techniques for making stable transgenic plants introduce foreign DNA into the plant nucleus. Foreign genes integrated into nuclear chromosomes are widely dispersed via pollen. Organelles, such as plastids and mitochondria, are maternally inherited in many crop plants. The introduction of foreign DNA into organelles provides one method for reducing transgene escape into the environment

Methods to introduce foreign DNA into organelles were developed after the advent of nuclear transformation technologies. Early described methods for transforming plastids were inefficient, not reproducible and of little value. Reproducible DNA-mediated transformation methods for organelles were first described for Chlamydomonas reinhardtii and Saccharomyces cerevisiae mitochondria. Subsequently a reliable procedure for stable plastid transformation of tobacco has been described.

The techniques generally used to introduce genes into plastids include particle bombardment, polyethylene glycol and micro-injection. Such techniques are not 100% effective. To determine that the plant has been transformed, the gene of interest is introduced along with a selectable marker. The most commonly used selectable markers are those which confer resistance to an antibiotic to the transformed plant. The presence of the antibiotic resistance gene in the transformed plant allows a worker to differentiate and select transformed plants from wild-type, untransformed plants.

The developments in plastid transformation technology in land plants over the last decade have relied on the use of the bacterial aadA gene for plastid transformation, which confers resistance to spectinomycin and streptomycin. Plants containing the aadA gene grow normally on media containing spectinomycin and/or streptomycin whereas wild-type plants not containing the aadA gene will grow on such media, but are bleached, allowing simple differentiation between transformed green plants and wild-type white plants.

Use of the aadA gene marker from plasmid R100.1 for plastid transformation was first described in C.reinhardtii. Subsequent use of the aadA gene from Shigella in tobacco plastid transformation led to dramatic improvements in efficiency. Other selectable marker genes, e.g. the kanamycin resistance gene Kan, have proven to be less efficient than aadA in plastid transformation although it is envisaged that further selectable markers may be developed with equivalent, if not greater, efficiency than aadA.

There is considerable anxiety on the health and environmental risks posed by the presence of antibiotic resistance genes in genetically engineered crop plants. Methods to remove antibiotic resistance and other selectable marker genes from transgenic plants, whilst retaining the genes of interest, are of considerable value.

Currently, there are two general methods for producing transgenic plants which do not contain genes for antibiotic resistance. In the first method, a selection regime that does not require antibiotics is used. For example, mannose or isopentenyl transferase can be used to select transgenic plants. In the second method, antibiotic resistance genes are removed from transgenic plants after their production. A number of schemes for removal of selectable marker genes from chromosomes have been described. If the selectable marker gene is not closely linked to the gene of interest the marker may be removed by standard crossing and analysis of the progeny. When the selectable marker gene is closely linked to the gene of interest other schemes have been devised to ensure its removal. These include the use of transposable elements or site-specific recombination systems. These schemes are restricted to nuclear genes and are not relevant to removing selectable marker genes from transgenic plants containing modified plastid genomes.

In the alga C.reinhardtii, selection schemes based on photosynthetic mutants have allowed the introduction of foreign genes of interest into the plastid genome without selectable marker genes such as aadA. Such schemes are not practical in higher plants since they rely on the prior availability of photosynthetic mutants. A number of methods for modifying plastid DNA without the integration of foreign non-plastid genes, including selectable marker genes, have been reported. A shuttle vector system (NICE1) has been described in tobacco that allows engineering of plastid genes without concomitant integration of a foreign selectable marker gene. The system has allowed the replacement of endogenous plastid sequences with foreign plastid DNA sequences. NICE1-based plasmids are also suitable for rescuing mutations from any part of the plastid genome.

Schemes for the removal of the aadA gene from the plastid genome of C.reinhardtii have been described. Marker recycling in C.reinhardtii chloroplasts provides a method for the stepwise introduction of mutations into the C.reinhardtii plastid genome. Neither marker recycling nor the tobacco shuttle vector system have allowed the introduction of foreign genes of interest, which are not homologous to plastid genes, into the plastid genome.

There is a need to develop methods that allow the insertion of exogenous or foreign genes, which do not have a selectable phenotype, into the plastid genome, without the long-term integration of antibiotic resistance genes.

Methods for introducing foreign genes of interest into the plastid genome without the concomitant insertion of a selectable marker gene have not been described in higher plants. Such methods would have great utility in reducing the perceived environmental and health risks of transgenic plants by the general public. Methods that enable the use of a wide range of marker genes, which are too inefficient for current plastid transformation procedures involving direct selection, would also have widespread application in extending the range of transplastomic plants that can be produced.

According to the present invention in a first aspect there is provided a method for producing a transgenic plant comprising a recombinant plastid genome containing an exogenous gene in the absence of a selectable marker gene introduced with the exogenous gene, the method comprising:

(a) stably transforming the plastid genome of a plant cell with nucleic acid comprising an exogenous gene, a selectable marker gene and at least two direct repeat sequences arranged to effect a recombination event within the transformed plastid genome to excise the selectable marker gene, whilst retaining the exogenous gene;

(b) selecting for transformed plants whose plastids comprise the selectable marker gene on a first selection medium; and

(c) growing the selected transformed plants in on the absence of the first selection medium to promote excision of the selectable marker gene by recombination within the transformed plastid genome whilst retaining the exogenous gene.

The first aspect of the invention provides a method for producing transgenic plants that contain foreign genie(s) of interest within the plastid genome without selectable marker genes. The method involves the introduction of exogenous gene(s) of interest and a selectable marker gene into the plastid genome of plants. Once transplastomic plants are produced, the undesirable selectable marker gene is eliminated from the plastid genome. The invention in its first aspect provides a method for removing the undesirable foreign antibiotic resistance genes from a plant whose plastid genomes have been transformed with one or more desirable genes. Removal of undesirable genes has considerable value in reducing public concern over the escape of antibiotic resistance genes to other plants and the transfer of antibiotic resistance genes to bacteria.

Plants:

The method according to the first aspect of the invention is applicable to any multicellular plant into whose plastid it is desired to introduce an exogenous gene. The method is particularly applicable to tobacco, as plastid transformation systems for tobacco have been developed. However, the method according to the first aspect of the invention is also applicable to other higher plants especially those for which plastid transformation methods are being developed such as the cereals, the Brassicaceae and other Solanaceae species such as potato. It is envisaged that the method according to the first aspect of the invention will be applicable to monocots and dicots, including tree and conifers, as well as crop plants

Transformation:

There are a number of methods available for stably transforming higher plant plastids with foreign DNA and it is not intended that the method of the invention is restricted to any one of these methods. The most generally used transformation methods include particle bombardment, polyethylene-mediated transformation and micro-injection. The particular method chosen to obtain transformed plants containing plastid genomes with the inserted exogenous genes will depend on the plant species and organs chosen. In the examples described below plastid transformation vectors were introduced into tobacco leaves by particle bombardment.

The term “stably transforming the plastid genome of a plant cell with nucleic acid” means that under desired conditions the transformed plant cell retains the transfected phenotype and does not revert back to the wild-type. It is preferred that the transformed cells will be maintained in such a manner so as to allow a state of homoplasmy to be achieved following transfection, and the desired conditions are any in which the transformed cell can survive and which exert a selective pressure favoring growth and multiplication of transformed genomes, plastids and cells.

Furthermore, as used herein, the term “stably transforming the plastid genome of a plant cell with nucleic acid” means that subsequent to transformation the plastid genome contains non-native nucleic acid; the term is intended to imply nothing as to whether transformation occurred as a result of recombination of a single nucleic acid into the plastid genome or a plurality of nucleic acids into the plastid genome.

For stable plastid transformation nucleic acids containing a selectable marker gene and gene(s) of interest are inserted into the plastid genome by homologous recombination with plastid DNA sequences that are flanking introduced foreign genes and target the foreign genes to specific locations in the plastid genome. These plastid targeting regions are taken from clone banks of plastid DNA that are available for a large number of plants. For example clone banks containing plastid DNA restriction fragments are available for tobacco and barley. The precise integration of foreign genes within plastid DNA is facilitated by the complete sequences of an increasing number of plastid genomes, for example the plastid genomes of tobacco, rice and maize. In the examples discussed below foreign genes are inserted at position 59319 corresponding to an AocI restriction site of the tobacco plastid genome in the intergenic region between the rbcL and accD genes.

In practice, any nucleic acid used to transform plant cells will be in the form of a nucleic acid construct.

In practice, a construct used to transfect the plastid genome will additionally comprise various control elements. Such control elements will preferably include promoters (e.g. 16S rRNA promoter rrnBn and rrnHv) and a ribosome binding site (RBS), e.g. that derived from the tobacco rbcL gene, positioned at an appropriate distance upstream of a translation initiation codon to ensure efficient translation initiation. A chloroplast promoter is preferred. The Brassica napus chloroplast 16S rRNA promoter and Hordeum vulgare 16S rRNA promoter used in combination with the 3′ regulatory region of the plastid psbA gene provide two examples of preferred control elements. The invention is not restricted to these 5′ and 3′ regulatory sequences and numerous other bacterial or plastid promoter and 3′ regulatory regions may also be used.

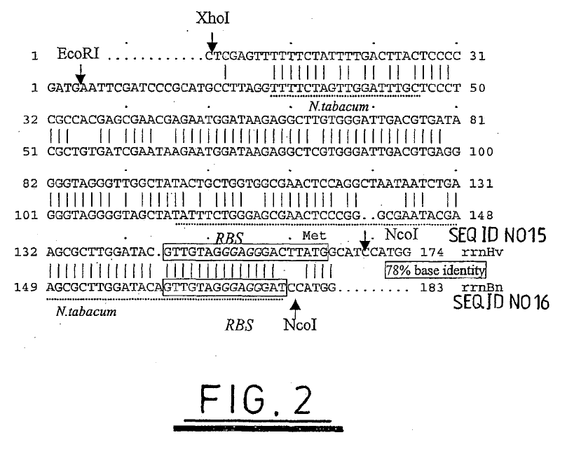

Preferred promoter sequences are shown in FIG. 2 as SEQ ID NO. 15 (rrnHv) and SEQ ID. NO. 16 (rrnBn) with EMBL/DDBJ/GenBank accessions AJ276676 and AJ276677.

According to an embodiment of the first aspect of the invention, the plastid genome is transformed with a nucleic acid construct comprising an expression cassette including an exogenous gene, a selectable marker gene and at least two direct repeat sequences. The transfected construct comprising the expression cassette incorporates into the plastid genome through homologous recombination events.

According to an alternative embodiment of the first aspect of the invention, plant cells transformed according to the first aspect of the invention may have been previously transformed with one or more genes or may be subsequently transformed with one or more genes to bring about the method of the first aspect of the invention. In other words, rather than transforming the plastid genome with a single construct comprising an expression cassette including an exogenous gene, a selectable marker gene and at least two direct repeat sequences arranged to effect a recombination event within the transformed plastid genome to excise the selectable marker gene, whilst retaining the exogenous gene, nucleic acid comprising an exogenous gene may be transformed into the plastid genome separately from the selectable marker gene and direct repeat sequences.

The nucleic acid comprising the exogenous gene may be transformed into the plastid genome on a construct comprising an expression cassette for the selectable marker gene and direct repeat sequences. Alternatively, the plastid genome may be transformed with separate nucleic acid sequences, one comprising the exogenous gene, another comprising the selectable marker gene and direct repeat sequences.

When two nucleic acid sequences are used they are preferably introduced together by co-transformation.

Exogenous Gene:

The exogenous gene introduced into the plastid genome in accordance with the method of the first aspect of the invention may be any gene which it is desired to introduce into a transgenic plant. The benefits of inserting exogenous genes into the plastid genome of plants are great. Desirable genes or genes of interest confer a desirable phenotype on the plant that is not present in the native plant. Genes of interest may include genes for disease resistance, genes for pest resistance, genes for herbicide resistance, genes involved in specific biosynthetic pathways or genes involved in stress tolerance. The nature of the desirable genes is not a critical part of this invention.

Selectable Marker:

The selectable marker used in accordance with the method of the first aspect of the invention is preferably a nonlethal selectable marker that confers on its recipients a recognizable phenotype. Commonly used selectable markers include resistance to antibiotics, herbicides or other compounds, which would be lethal to cells, organelles or tissues not expressing the resistance gene or allele. Selection of transformants is accomplished by growing the cells or tissues under selective pressure, i.e. by on media containing the antibiotic, herbicide or other compound. If the selectable marker is a “lethal” selectable marker, cells expressing the selectable marker will live, while cells lacking the selectable marker will die. If the selectable marker is “non-lethal”, transformants will be identifiable by some means from non-transformants, but both transformants and non-transformants will live in the presence of the selection pressure.

A selectable marker may be non-lethal at the cellular level but lethal at the organellar level. For example the antibiotic spectinomycin inhibits the translation of mRNA to protein in plastids, but not in the cytoplasm. Plastids sensitive to spectinomycin are incapable of producing proteins required for plastid survival, and the tissues of a plant grown on spectinomycin are bleached white, instead of being green. Tissues from plants that are spectinomycin resistant are green. In a mixed population of cells containing transformed and non-transformed plastids, the sensitive non-transformants will disappear during the course of plastid/cell division under selection pressure, and eventually only transformed plastids will comprise the plastid population. When a plant contains a uniform population composed of only one type of plastid genome it is said to be homoplasmic. Selection produces homoplasmic plants, which only contain transformed plastid genomes.

A preferred selectable marker according to the first aspect of the invention is the aadA selectable marker, winch confers resistance, to spectinomycin and/or streptomycin. The use of other efficient selectable markers is also envisaged.

Selective Gene Excision From Recombinant Plastid Genomes:

Excision of the selectable marker gene is mediated by recombination events between repeated DNA sequences. This can be mediated by native plastid recombination enzymes or foreign site-specific recombination enzymes. Plastids contain an efficient homologous DNA recombination pathway that allows the precise targeting of foreign DNA into the plastid genome. In addition, evolutionary comparisons between plastid DNA from different species and studies on mutants suggest that plastids are endowed with the necessary replication and recombination enzymes to mediate alterations involving short directly repeated DNA sequences. DNA slippage during replication provides one mechanism for allowing changes in repeat number and length for short repeats which can be a few base pairs in length. Recombination between DNA sequences also provides a mechanism for altering the sequence organization of plastid genomes. This has been deduced from comparative studies on plastid genomes from different species, analysis of plastid DNA mutants and by studying plastid transformants.

Analyses of plastid transformants in tobacco suggest DNA recombination events between repeated sequences as short as 393 bp and 950 bp. Evolutionary studies suggest recombination events between much shorter DNA direct repeats of less than 20 base pairs can take place in plastids. Although, these evolutionary studies do not provide information on the frequency of these recombination events they do imply that plastids contain the necessary machinery to recognize and recombine very short directly repeated DNA sequences.

A direct repeat sequence is a nucleic acid sequence that is duplicated in the construct and the duplicated nucleic acid sequences are directly orientated rather than inversely orientated.

The direct repeat may comprise any nucleic acid sequence including regulatory sequences that normally flank coding sequences. The direct repeat may comprise foreign nucleic acid sequences with little similarity to the plastid genome being transformed. Indeed this is preferred, to lessen the opportunity of recombination between an inserted sequence and an endogenous sequence of the plastid occurring.

It is proposed that the frequency of selectable marker excision will be related to the length of the directly repeated DNA sequences. The length of sequence that is directly repeated to form a direct repeat is at least 20 nucleotides, preferably at least 50, and most preferably at least 100 nucleotides. It is envisaged that the longer the direct repeat sequence, the more efficient the recombination event. In the examples, direct repeats as short as 174 bp have been shown to be effective in excision of the selectable marker gene. In another example a direct repeat sequence of 418 bp is used.

Thus it is proposed that the efficiency of the method according to the first aspect of the invention may be modulated by altering the length of directly repeated sequences. Although it is expected that the longer the length of the direct repeat sequence the more efficient the excision, it is preferred that the direct repeat sequences are less than 10,000 bp in length, more preferably less than 5,000 bp in length and most preferably less than 2,000 bp in length to facilitate cloning; the total size of foreign DNA being inserted into the plastid genome being an important factor in carrying out the invention.

It is further proposed that the efficiency of the method according to the first aspect of the invention may be modulated by altering the number of directly repeated sequences.

The introduction of several directly repeated DNA sequences into plastid transformation constructs containing three or more genes provides a particularly effective method for promoting selectable marker gene loss whilst retaining one or more gene(s) of interest.

The positioning of directly repeated DNA sequences in a multiple gene construct provides control over the relative excision frequency of genes from recombinant plastid genomes.

In an arrangement where nucleic acid to be introduced into a plastid genome comprises just the exogenous gene and selectable marker gene (along with any control elements) the direct repeats are preferably positioned to flank the selectable marker gene, i.e. two direct repeats are used.

In an arrangement where nucleic acid to be introduced into a plastid genome comprises more than one exogenous gene and selectable marker gene (along with any control elements) the direct repeats are preferably positioned to flank the selectable marker gene if both exogenous genes are required in the recombinant plastid genome. A single plastid transformation vector containing a selectable marker gene and multiple exogenous genes can also be used to excise the selectable marker gene and one or more exogenous genes whilst retaining the exogenous gene of interest. This is done by using sets of directly repeated sequences whose borders flank the selectable marker gene and one or more exogenous genes. When the selectable marker gene is positioned centrally between two exogenous genes, two sets of direct repeats are located to promote loss of the marker gene and a single exogenous gene. One set of direct repeats promotes loss of the marker gene plus the left exogenous gene. The second set of direct repeats promotes loss of the marker gene plus the right exogenous gene. These excision events allow the production of two different marker-free plastid genomes from a single plastid transformation construct. These recombinant plastid genomes are of two types: they either contain the exogenous gene located to the left of the marker gene or they contain the exogenous gene located to the right of the marker gene in the original construct.

Selection of Transplastomic Plants:

After transformation integration of foreign DNA into the plastid genome is selected using media containing the marker to which resistance is conferred by the selectable marker gene. Using as an example, the aadA gene as a selectable marker gene, the selection medium contains spectinomycin or streptomycin. This first round of selection produces plant clones and material capable of growth on medium containing spectinomycin or streptomycin. These clones and material are propagated under spectinomycin or streptomycin selection until homoplasmic plants are produced in which all plastid genomes in a plant contain a foreign insert.

Excision of Undesirable Genes:

Once homoplasmy of recombinant plastid genomes is achieved, selection for the selectable marker gene is removed in To plants and their progeny. The removal of selection promotes the loss of the selectable marker gene. Loss of the selectable marker gene may be monitored by sensitivity of plants to the first selection medium and molecular techniques such as Southern blot hybridization and the polymerase chain reaction.

As described above the method according to the first aspect of the invention may be used to introduce an exogenous gene into a plastid genome, allow selection of transformed plants using a selectable marker and yet provide for excision of the selectable marker so as to allow the transgenic plants produced to be acceptable to the public.

A preferred embodiment of the first aspect of the present invention will now be described in relation to producing a transgenic tobacco plant comprising a recombinant plastid genome containing an exogenous uidA gene (encoding β glucuronidase) in the absence of the aadA gene introduced with the uidA gene.

In a typical method according to the first aspect of the invention, constructs containing an exogenous gene and an aadA gene are used to transform tobacco plants by particle bombardment. Typically bombarded organs are cultured as small pieces on solid media containing plant hormones for 40-72 hours to recover. They are then transferred onto selective medium containing spectinomycin and streptomycin, which allows resistant cells to grow and divide. Resistant material such as green shoots and green callus are subcultured on media containing spectinomycin and streptomycin. Shoots are subcultured until homoplasmy of recombinant plastid genomes is reached. Plants are then transferred to soil and the young leaves and apical meristem sprayed with a solution of spectinomycin and streptomycin for a period of 2-3 weeks.

The first aspect of the invention has been described above in relation to using a strongly selectable marker. Selectable markers, which result in poor plastid transformation frequencies are not widely used in current plastid transformation methods. In these cases, the present invention allows these marker genes, which confer for example resistance to an antibiotic or herbicide, to be used for plastid transformation in a two step selection procedure in which a strong selectable marker (e.g. the aadA gene) is used first. Dual selection provides a powerful screen for potential plastid transformants. It greatly increases the probability of isolating genuine plastid transformants from the background of non-transformed plants. The utilization of a greater variety of selective agents to select plastid transformants will be particularly beneficial where an existing selective agent has been shown to be particularly efficient for a plant species recalcitrant to DNA-mediated transformation. In addition, the use of a second selective agent provides flexibility when the continued exposure of a plant to spectinomycin or streptomycin is undesirable.

When using a two gene system, once the nucleic acid is transformed into plants the initial transformants are selected by growing on the selection medium for the strong selectable marker e.g. by growing on streptomycin/spectinomycin). Transformed cells are differentiated from untransformed cells by the property of the selectable marker. The initial transformants are then placed on a second medium which selects for the second selectable marker gene. Once selection has been initiated for the second selectable marker gene, the first selectable marker is no longer required and may be eliminated. Elimination of the first selectable marker is mediated by recombination between direct repeats that flank it. The stochastic processes of plastid DNA replication and segregation during, cell division (cytoplasmic sorting) together with gene excision will produce homoplasmic plants that only contain the second marker gene, for example a herbicide resistance gene.

Agents that promote DNA-mediated recombination events in plastids can be used to induce loss of the selectable marker gene. For example promotion of recombination in plastids by exposure to gamma irradiation leads to loss of the selectable marker gene by recombination between direct repeat sequences.

Accordingly the first aspect of the invention may further comprise stimulating DNA mediated recombination in plastids using specific proteins, chemical agents or physical agents such as gamma irradiation to promote excision of the selectable marker gene.

In the example, described below a modified bar gene was used to provide resistance to glufosinate-ammonium. The modified bar gene has a high guanine plus cytosine content of 68% which is not optimal for high level expression in the plastid. Use of this bar gene, or similar genes which might be expected to be weakly expressed in plastids, provides strong selection pressure for obtaining homoplasmic recombinant plastid genomes. Plants or plant cells containing the second selectable marker gene will have a distinctive phenotype for the purposes of identification to distinguish them from untransformed cells.

Therefore, according to an embodiment of the first aspect of the invention there is provided a method for producing a transgenic plant comprising a recombinant plastid genome containing an exogenous gene in the absence of a first selectable marker gene introduced with the exogenous gene, the method comprising:

(a) stably transforming the plastid genome of a plant cell with nucleic acid comprising an exogenous gene, a first selectable marker gene and a second selectable marker gene and at least two direct repeat sequences arranged to effect a recombination event within the transformed plastid genome to excise the first selectable marker gene, whilst retaining the exogenous gene;

(b) selecting for transformed plants whose plastids comprise the first selectable marker gene on a first selection medium; and

(c) growing the selected transformed plants on a second selection medium to allow selection of plants containing the second selectable marker gene to allow excision of the first selectable marker gene by recombination within the transformed plastid genome whilst retaining the exogenous gene.

This embodiment of the present invention allows transformation of a plastid with a gene of interest which confers a property that cannot normally be selected for.

It may be that the exogenous gene confers a property than can be weakly selected for. In such a situation it is not necessary to have two selectable marker genes, one of the selectable marker genes is provided by the exogenous gene.

In a situation when the gene of interest confers a property that cannot be selected it is important to select plants that are deficient in wild type plastid genomes and that only contain transformed plastid genomes with selectable marker genes such as an antibiotic resistance gene or herbicide resistance genes plus the gene of interest. Once homoplasmy of recombinant plastid genomes is reached selection is removed to enable excision of undesirable selectable marker genes whilst retaining the genes of interest. Continued propagation of cell lines and plants in the absence of selection will result in loss of the selectable marker genes and the generation of a recombinant plastid genome which only contains the genes of interest. Excision of selectable marker genes is promoted by the number of directly repeated sequences in a construct as well as the length of the repeats. Three directly repeated DNA sequences have proved particularly effective in the removal of two selectable marker genes whilst retaining the gene of interest. In the examples described below the uidA gene was used as the unselectable gene of interest.

In both cases, with either one or more selectable markers, integration at the correct site of the plastid genome and homoplasmy of recombinant plastid genomes is verified by Southern blot hybridization. In the examples provided below an 11.4 kbp HindIII fragment produced by native plastid DNA is replaced by new HindIII fragments containing one or more foreign genes.

In examples two genes of interest were used to illustrate the method according to the first aspect of the present invention. These were the bar gene from Streptomyces hygroscopicus (White et al., 1990) and the coding region for the uidA gene encoding β-glucuronidase from Escherichia coli (Jefferson et al., 1986).

The bar gene confers resistance to glufosinate-ammonium and is an example of a gene that confers a selectable property on plants. The bar gene was modified by PCR cloning for expression in plastids. This involved the introduction of a NcoI restriction site within its N-terminal coding region, the conversion of the second codon to glycine from serine and the insertion of two TAA termination codons. The β-glucuronidase gene can be detected by simple colorimetric or fluorimetric enzyme assays and is an example of a gene of interest that cannot be selected using antibiotics or herbicides. The invention is not restricted to these coding sequences and numerous other genes of interest may also be used.

According to the present invention in a second aspect there is provided a nucleic acid construct for transforming a plant plastid genome comprising at least two direct repeat sequences and a selectable marker gene. The structural features of the nucleic acid constructs according to the second aspect of the invention are detailed in the description of the first aspect of the invention. Such nucleic acid constructs can be made using standard techniques known in the art.

The nucleic acid construct of the second aspect of the invention may further comprise an exogenous gene and preferably may comprise a second exogenous gene.

In the nucleic acid construct of the second aspect of the invention the direct repeat sequence may be at least 20 nucleotides in length, preferably at least 50 nucleotides in length, more preferably at least 100 nucleotides in length, more preferably 174 nucleotides in length and most preferably is 418 nucleotides in length.

Generally, for ease of genetic manipulation it is preferred that the direct repeat sequence is less than 10,000 nucleotides in length.

Th direct repeat sequence preferably comprises a Ntpsb A sequence, especially that shown as SEQ ID NO.14.

The direct repeat sequence may comprise a rrnHv promoter sequence, such as shown as SEQ ID NO.15.

The direct repeat sequence may comprise a rrnBv promoter sequence, such as shown as SEQ ID NO.16.

The exogenous gene of the nucleic acid construct of the second aspect of the invention is preferably a gene for disease resistance, a gene for pest resistance, a gene for herbicide resistance, a gene involved in specific biosynthetic pathways or a gene involved in stress tolerance.

Preferably the exogenous gene is a uidA gene or a bar gene, preferably a modified bar gene shown as SEQ ID. NO. 17.

The selectable marker gene of the construct preferably encodes a selectable marker that is non-lethal. Such a selectable marker gene is the bacterial aadA gene.

The second exogenous gene of the construct may be a selectable marker gene, for example a bar gene such as the modified bar gene having the sequence shown as SEQ ID NO. 17.

In the nucleic acid construct of the second aspect of the invention the direct repeat sequences preferably flank the selectable marker. In a nucleic acid construct having two exogenous genes where it is desirable to excise one of the exogenous genes, the construct preferably comprises three direct repeat sequences, two flanking the selectable marker gene and one flanking one of the exogenous genes.

The nucleic acid constructs according to the second aspect of the invention may be incorporated into plasmids for transforming a plant plastid genome.

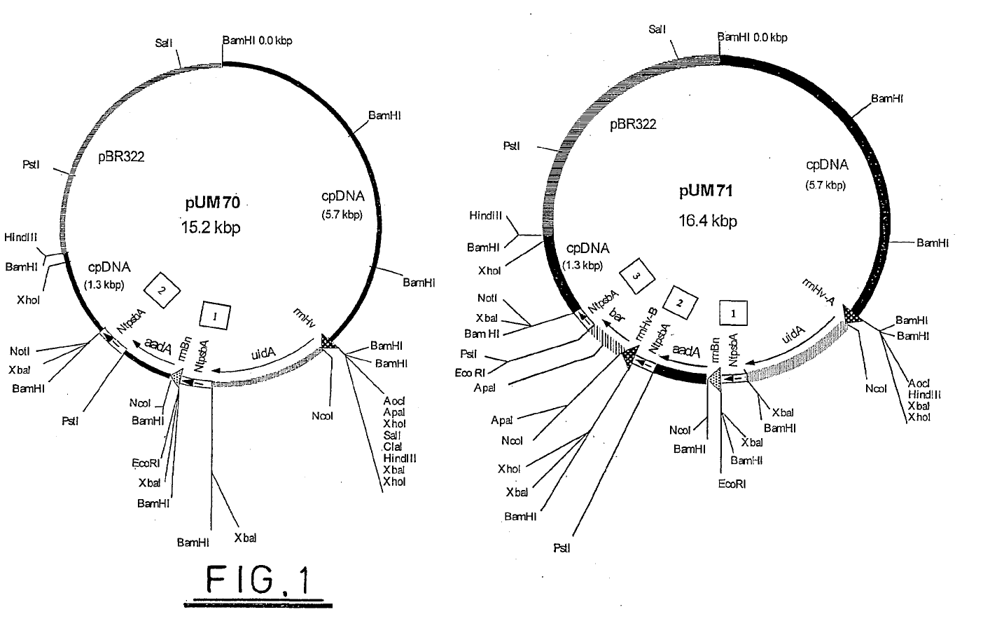

Preferred plasmids are pUM71 comprising the bar gene, the uidA gene, the aadA gene, three copies of a directly repeated sequence of NtpsbA, two copies of a directly repeated sequence of rrnHv and one copy of rrnBv and pUM70 comprising the uidA gene, the aadA gene and two copies of a directly repeated sequence of NtpsbA. Restriction maps of these two plasmids are provided in FIG. 1.

These plasmids may be used to transform plant plastid genomes according to the method of the first aspect of the invention.

As described in relation to the first aspect of the invention nucleic acid may be composed of plastid expression cassettes which comprise 5′ and 3′ regulatory regions. Coding sequences for proteins are inserted into expression cassettes. Expression cassettes with coding regions may be integrated into an intergenic region of previously cloned plastid DNA for targeting within the plastid. The complete construct is propagated in E. coli cloning vector such as pBR322, pAT153, vectors of the pUC series and pBluescript vectors. For the purposes of this invention excision of genes is controlled by the organization of directly repeated DNA sequences. The length and number of directly repeated sequences in a construct control the frequency of gene excision. The actual sequence of a directly repeated DNA element is not critical for the invention. Increasing the length of the foreign DNA sequence to be inserted into the plastid genome is also beneficial for promoting subsequent gene loss. When excision of a gene is not required it is important to reduce the length of any directly repeated sequences that flank it. This requires the utilization of non-redundant flanking DNA sequences which includes regulatory elements such as promoters and terminators. The genes of interest and aadA gene can be introduced as a single piece of DNA within the same construct or as separate constructs. The frequency of co-transformation of two unlinked genes, on separate plasmids, into the plastid genome is high.

In a third aspect the invention comprises a cell or cells and multicellular plant tissue preferably whole plants, calli and leaf tissue) having cells whose plastids comprise an exogenous gene but do not contain a selectable marker gene introduced with the exogenous gene.

The cells and plant tissue according to the third aspect of the invention are prepared according to the methods of the first aspect of the invention.

In a fourth aspect the present invention provides transgenic plants comprising an exogenous gene in their plastid genomes, produced according to the method of the first aspect of the invention.

The method of the first aspect of the invention is used to transform plastids of plant cells and then standard conditions are used to facilitate the reproduction, differentiation and growth of such cells into multicellular tissue.

Regeneration of intact plants may be accomplished either with continued selective pressure or in the absence of selective pressure if homoplasmy has already been achieved within the transformed cell line.

The transgenic plant can be monocotyledonous or dicotyledonous and the cells of the tissue photosynthetic and/or non-photosynthetic.

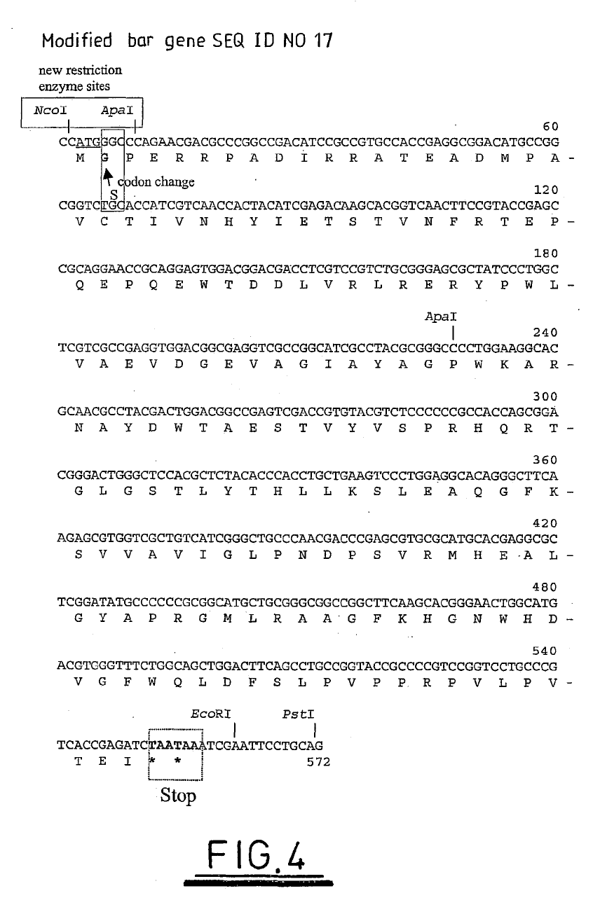

A preferred transgenic plant according to the fourth aspect of the invention is a transgenic tobacco plant containing the modified bar gene shown in FIG. 4 in its plastids. This transgenic plant is resistant to glufosinate ammonium.

Although described in relation to a selectable marker gene as being the gene introduced and then excised, the purpose of this invention is to remove undesirable foreign DNA sequences from the plastid genome of transplastomic plants. The presence of antibiotic resistance genes is nearly always undesirable in transformed plastid genomes. In most instances, the definition of what is an undesirable sequence is not fixed but will depend on the phenotype desired in the plant. For example, a gene that confers herbicide resistance may be desirable in some situations but not in others. If herbicide resistance is required in a plant then all foreign genes not needed for this purpose are eliminated. Alternatively, if the gene of interest relates to some other property then all other foreign genes including herbicide resistance genes and antibiotic resistant selectable markers are eliminated to leave the gene of interest. A plant that is “free of” foreign ancillary sequences is one in which the undesired sequences are not detectable by Southern blot hybridization.

The present invention will now be described, by way of example only, with reference to the following drawings in which:

FIG. 1 shows restriction maps of plastid transformation vectors pUM70 & pUM71;

FIG. 2 shows the sequence and comparison of plastid promoters rrnHv (SEQ ID. NO. 15) and rrnBn (SEQ ID NO. 16); rrnHv contains a modified 16S rRNA promoter of barley plastid DNA fused to the ribosome binding site (RBS) and initiating ATG codon of the barley rbcL gene. The promoter is most suitable for monocotyledonous plants such as cereals. The 16SrRNA promoter region of rrnBn contains Brassica napus plastid DNA sequences fused to modified Nicotiana tabacum plastid DNA sequences. This chimeric 16S rRNA promoter region is fused to the RBS of the N. tabacum rbcL gene. N.tabacum sequences are underlined, bases 46-116 are from B. napus. The promoter is most suitable for dicotyledonous plants.

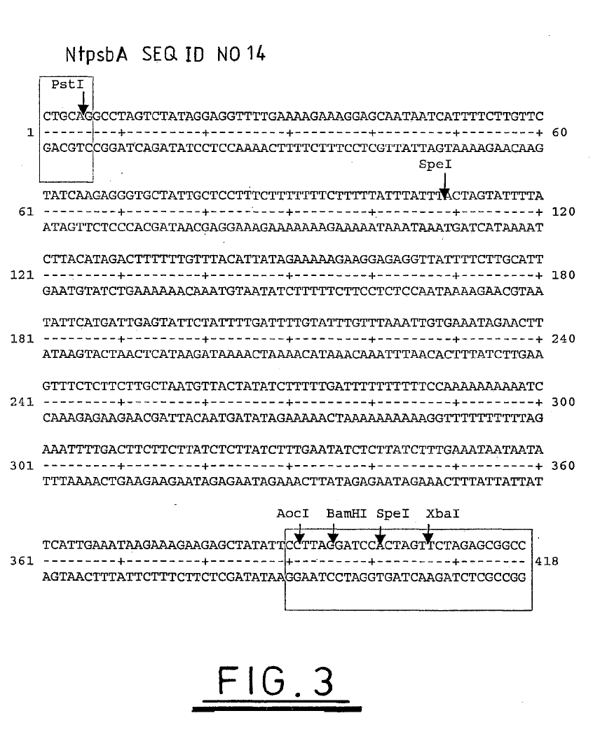

FIG. 3 shows the sequence of the 3′processing/terminator region of NtpsbA (SEQ ID NO. 14). The terminator region of the N tabacum psbA gene was modified by the insertion of an upstream Pst I site and downstream AocI and BamHI Sites to facilitate cloning into plastid expression cassettes.

FIG. 4 shows the sequence of the modified bar gene (SEQ ID NO. 17). The bar gene (White et al., 1991) was modified at the N and C terminus to enable its expression within the plastid using the plastid regulatory sequences described in FIGS. 2 and 3. The modifications introduce a NcoI site at its N-terminus and two TAA stop codons at the C-terminus. The second amino acid of the bar gene was changed from serine to glycine.

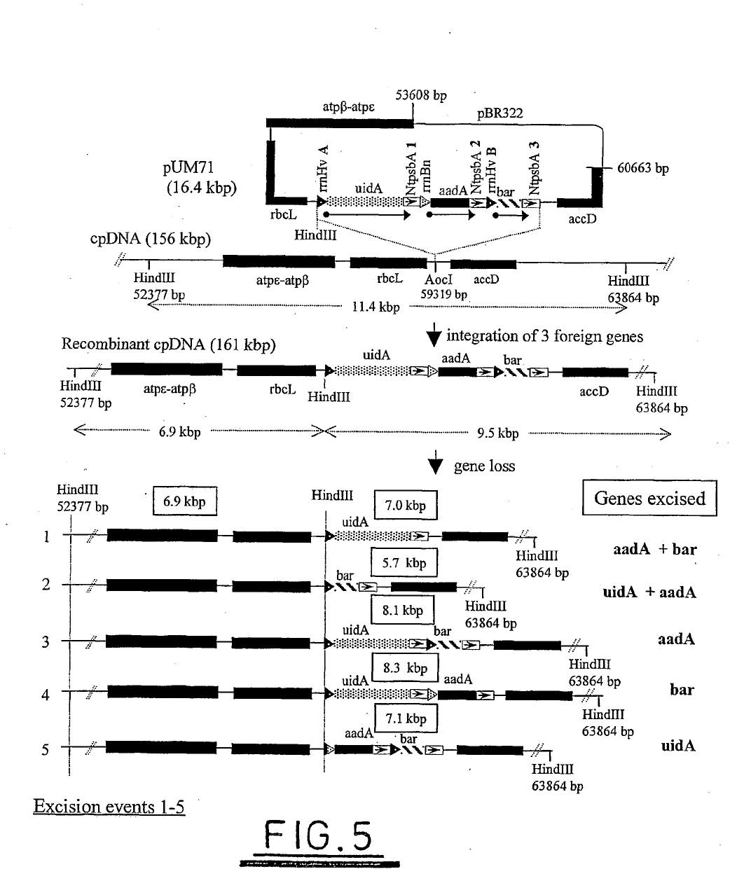

FIG. 5 illustrates a scheme for integration of pUM71 cassette into the plastid genome and genie-loss mediated by recombination events. Integration of the intact 4.9 kbp insert containing the uidA, aadA and bar genes into the plastid genome produces a recombinant plastid genome of 161 kbp. Selection for the bar gene using glufosinate-ammonium ensures the replacement of native plastid genomes by recombinant genomes containing the bar gene. The length and placement of directly repeated DNA sequences controls the frequency and types of genes lost. In pUM71 plastid transformants, selection for the bar gene is compatible with aadA and uidA gene loss mediated by recombination events between rrnHv A and rrnHv B (Case 2). Recombination between NtpsbA 1 and NtpsbA 3 excises aadA and bar (Case 1). Recombination between NtpsbA 1 and NtpsbA 2, or rrnBn and rrnHv B excises aadA (Case 3). Recombination between NtpsbA 2 and NtpsbA excises bar (Case 4). Recombination between rrnHv A and rrnBn excises uidA (Case 5). Cases 1 and 2 produce plastid genomes only containing a gene of interest which is either bar or uidA. Recombination between rrnHv A and B would not be expected at high frequency.

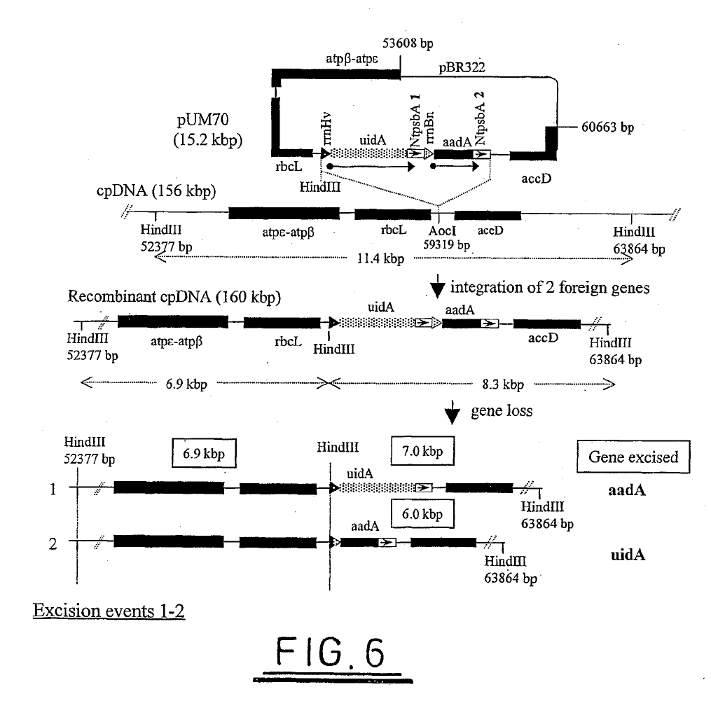

FIG. 6 illustrates a scheme for integration of pUM70 cassette into the plastid genome and gene-loss mediated by recombination events. Integration of the intact 3.8 kbp insert containing the uidA and aadA genes into the plastid genome produces a recombinant plastid genome of 160 kbp. Selection for the aadA gene using spectinomycin and streptomycin ensures the replacement of native plastid genomes by recombinant genomes containing the aadA gene. Once homoplasmy of recombinant aadA containing genomes is achieved selection pressure is removed. Excision of aadA is mediated via recombination between the two NtpsbA direct repeats (Case 1). Excision of uidA would be mediated by recombination between rrnHv and rrnBn imperfect direct repeats (Case 2) and would not be expected at high frequency. Case 1 leads to the generation of recombinant plastid genomes only containing uidA.

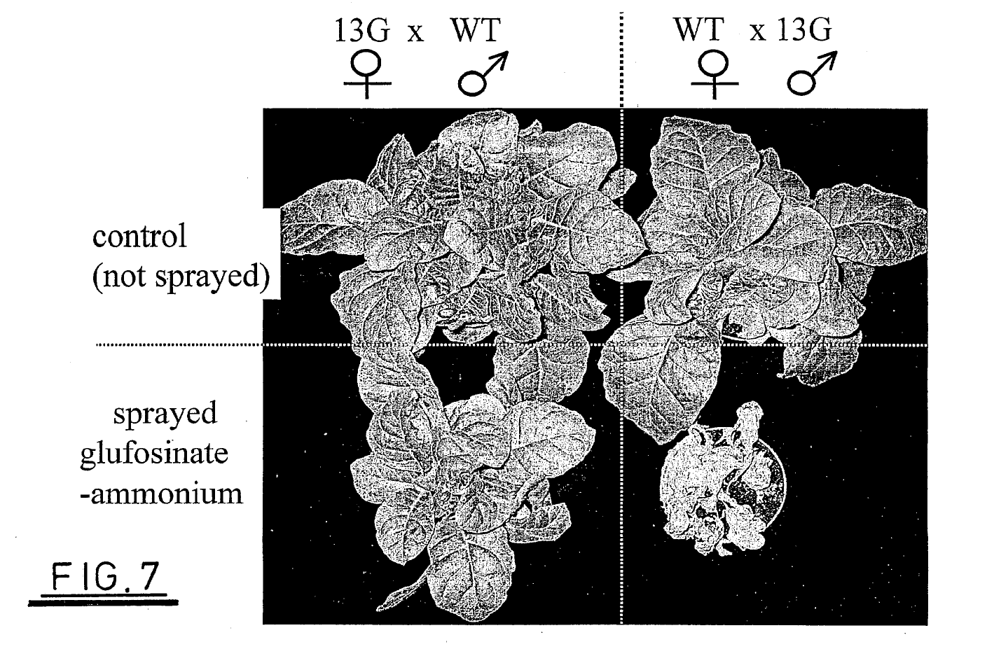

FIG. 7 shows the maternal inheritance pattern of glufosinate-ammonium resistance in pUM71 transplastomic plants. Reciprocal crosses were conducted in which flowers on the pUM71 transformant 13G was used as both the pollen donor and acceptor sites in crosses with flowers on untransformed wild type (WT) tobacco plants. In the cross, pUM71-13G (female)×WT (male) all progeny have the glufosinate-ammonium resistance phenotype of the maternal 13G parent. In the cross, WT (female)×pUM71 (male) all progeny have the glufosinate-ammonium sensitive phenotype of the maternal WT (untransformed) parent. Control plants are compared with plants sprayed with a 0.1% (V/V) solution of Challenge (AgrEvo, 150 g/l glufosinate-ammonium) on days 36, 43 and 50 following planting. Pots were photographed on day 57. Each pot contained five plants.

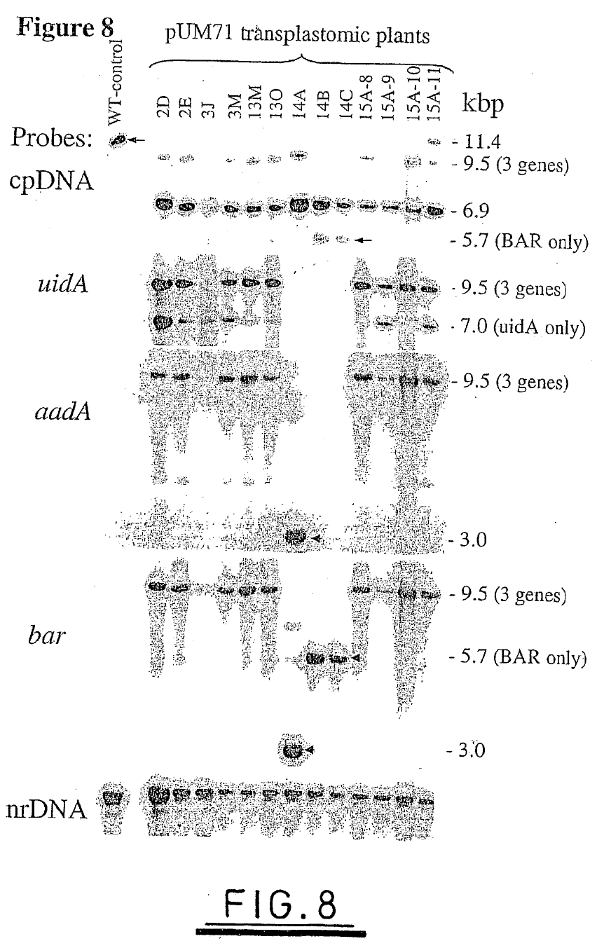

FIG. 8 shows Southern blot analyses of primary pUM71 transformants (T₀ generation) illustrating gene loss and production of aadA-free transplastomic plants containing the bar gene. HindIII digested total DNA (2 μg) from individual plants probed with: cpDNA flanking the insertion site (3.4 kbp ClaI-EcoRV fragment spanning bases 57176 to 60604 of the N. tabacum plastid genome, uidA, aadA and bar. A nuclear ribosomal DNA from B. napus was used to monitor similar DNA loading per lane. The 9.5 kbp HindIII band hybridizes to all three genes (uidA, aadA and bar). The 7.0 kbp HindIII band only hybridizes to the uidA probe. The 5.7 kbp HindIII band only hybridizes to the bar gene. The sizes and hybridization patterns of the 7.0 and 5.7 kbp bands are the outcome of recombination event shown in FIG. 5 (Cases 1 and 2). Plants 14A and 14B do not contain any detectable aadA or uidA, sequences and are glufosinate-ammonium resistant but spectinomycin-sensitive. Blots were hybridized at 60° C. and washed in 0.1% SSC, 0.1% SDS at 60° C. Sizes of restriction fragments were estimated from DNA size markers.

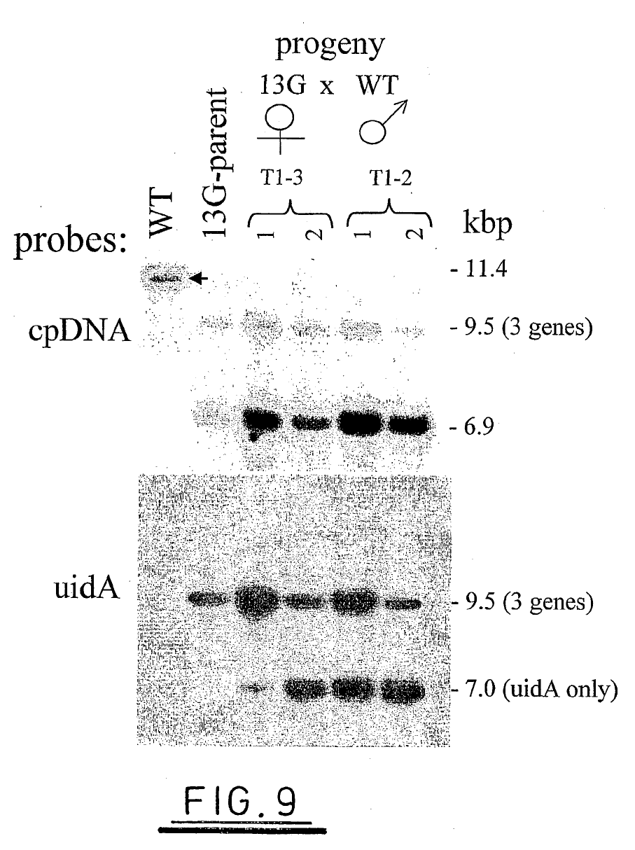

FIG. 9 shows Southern blot analyses of progeny of pUM71 transformant (Ti generation) illustrating aadA gene loss during propagation. Total DNA was prepared from separate leaf areas of two T2 progeny (2 and 3) of a 13G (female)×WT cross (male). HindIII digests of progeny and parental DNA probed with: (a) cpDNA flanking insertion site, (b) uidA. Blots were washed in 0.1% SSC, 0.1% SDS at 60° C.

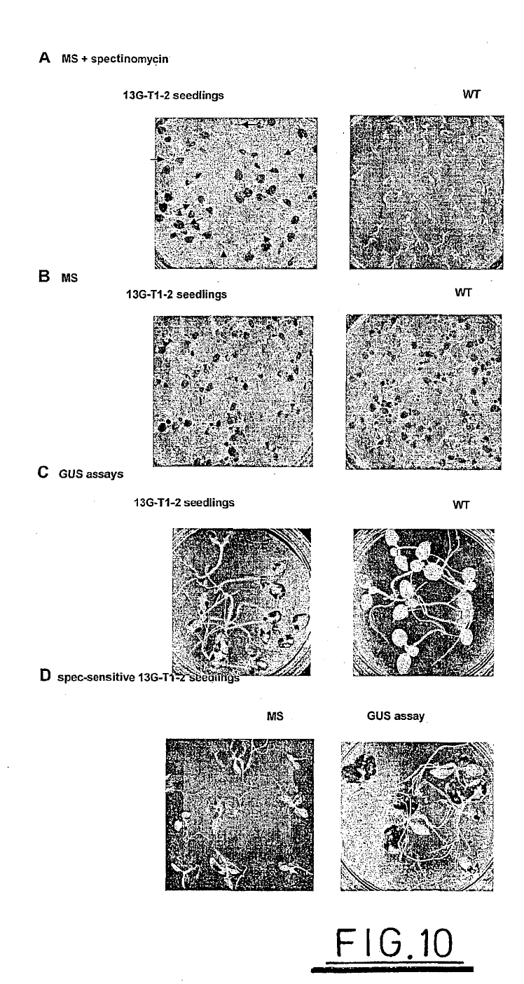

FIG. 10 shows marker-free plastid transformants containing the uidA gene. Seeds (T₂) from transplastomic plant 13G-T1-2 and control WT plants were surface-sterilised and plated on (A) MS salts medium containing 500 mg/ml spectinomycin (bleached seedlings from parent 13G-T1-2 are arrowed) or (B) MS salts medium. (C) β-glucuronidase (GUS) activity in green WT and spectinomycin-resistant seedlings from parent 13G-T1-2. (D) Re-greening of bleached 13G-T1-2 seedlings on MS salts medium lacking spectinomycin allows detection of GUS activity. These spectinomycin-sensitive seedlings containing the uidA gene are marker-free transplastomic seedlings. GUS is the product of the uidA reporter gene and converts X-Gluc to a blue product, which appears as darkly stained leaves in contrast to the white GUS negative wild-type seedlings.

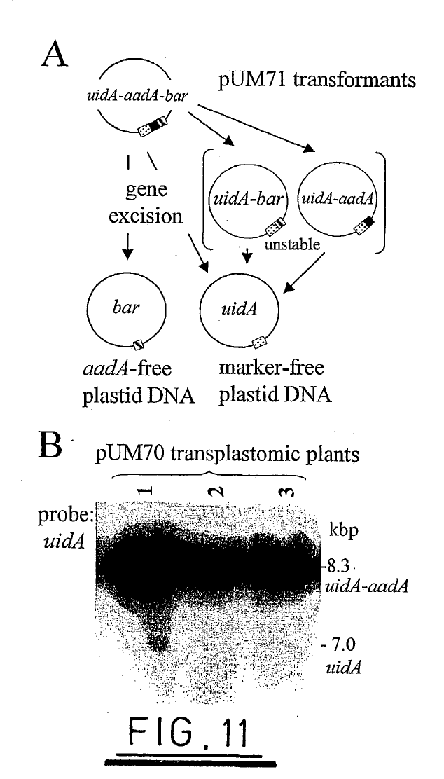

FIG. 11 shows the generation of aadA-free and marker-free plastid genomes from pUM71 plastid transformants. (A) Transplastomic pUM71 transformants containing the uidA, aadA and bar genes generate either aadA-free plastid genomes containing the bar gene or marker-free plastid genomes containing the uidA gene. Only one recombination event between the two 174 base direct repeats is possible and this produces aadA-free plastid genomes containing the bar gene. Three recombination events are possible between the three 418 bp NtpsbA repeats in pUM71 transformants to produce genomes containing uidA alone, uidA-bar or uidA-aadA. Plastid genomes uidA-bar and uidA-aadA, which contain two NtpsbA repeats, do not accumulate to high levels. If these genomes are produced they are unstable due to recombination between the remaining NtpsbA direct repeats. (B) Southern blot of DNA from three pUM70 transformants probed with uidA. Blot washed in 0.1% SSC, 0.1% SDS at 60° C. The 8.3 kbp Hind III band containing tandem uidA and aadA genes is diagnostic of the uidA-aadA plastid genome shown in (A). This shows that the uidA-aadA intermediate in (A) is not intrinsically unstable.

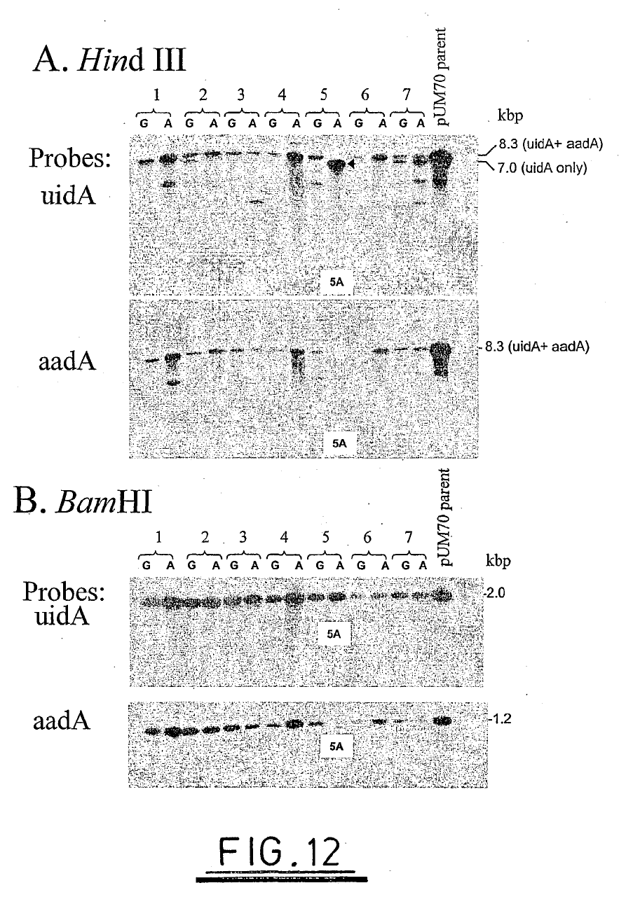

FIG. 12 shows Southern blot analyses of irradiated progeny of pUM70 transformants (T1 generation) illustrating gene loss and production of an aadA-free transplastomic plant containing the uidA gene. After irradiation plants from individual seeds exhibited green/white variegation. Green (G) and albino (A) shoots produced wholly green and white plants that were propagated separately. DNA from green and white plants derived from the same seed were analysed in adjacent lanes on blots. 2 μg of total DNA was digested with either HindIII or BamHI and probed with uidA and aadA specific probes as indicated. The 8.3 kbp band contains both uidA and aadA genes and is present in the majority of plants. In the plant 5A this 8.3 kbp band is replaced by a band of 7.0 kbp which only hybridizes to uidA. Plant 5A does not contain any detectable aadA sequences and is spectinomycin sensitive. It is an example of a “marker-free” transplastomic plant. The 7.0 kbp Hind III band, containing uidA only, is derived from recombination between the two NtpsbA repeats (FIG. 6, Case 1).

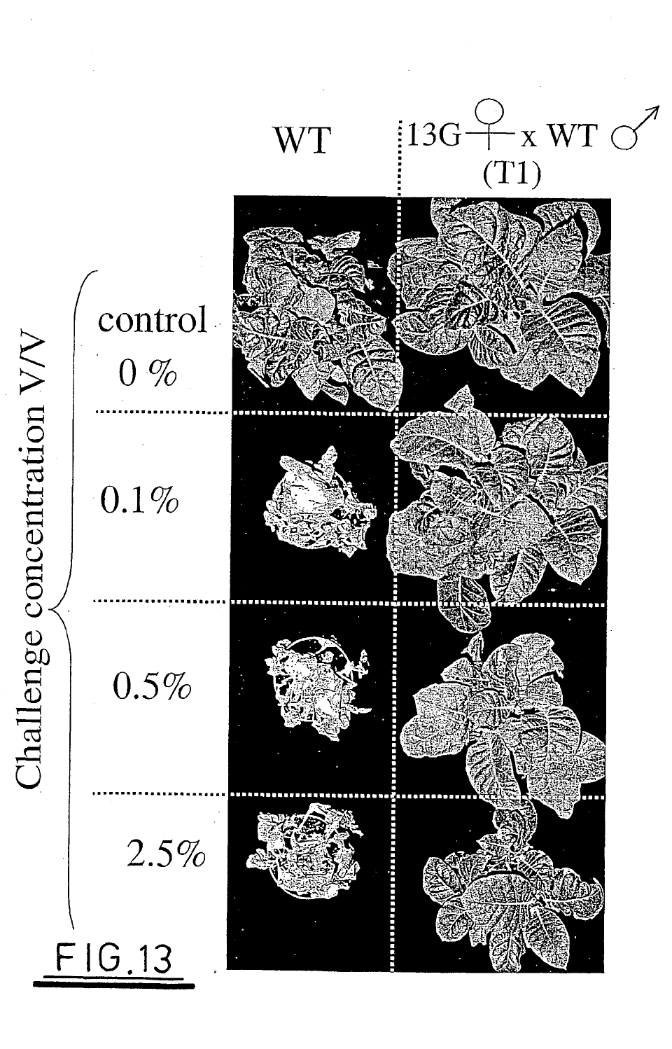

FIG. 13 shows Glufosinate-ammonium tolerance of transplastomic tobacco plants transformed with pUM71. Control untransformed plants and pUM71 transplastomic plants, T2 progeny of 13G (female)×WT cross (male), sprayed at 45, 49 and 53 days following planting with 0%, 0.1%, 0.5% and 2.5% (V/V) solutions of Challenge TM and photographed on day 71. Each pot contained four plants

### EXAMPLES

### General Methods

The laboratory procedures described below for manipulating and detecting recombinant DNA are those well known and commonly employed in the art. Standard techniques are used for cloning, nucleic acid isolation, amplification and purification and are described in Sambrook et al., Molecular Cloning-A Laboratory Manual, Cold Spring Harbor Laboratory, Cold Spring Harbor, N.Y. (1989). Enzymatic reactions involving DNA ligase, DNA polymerase, restriction enzymes were performed according to the manufacturers' specifications.

In the experimental disclosure which follows, all temperatures are given in degrees centigrade (° C.), weights are given in grams (g), milligrams (mg) or micrograms (μg), concentrations are given in molar (M), millimolar (mM) or micromolar (μM) and all volumes are given in liters (l), milliliters (ml) or (microlitrers (μl), unless otherwise indicated.

### Example 1

### Plastid Transformation Vectors

The restriction maps of the pUM70 and pUM71 plastid transformation vectors are shown in FIG. 1. In FIG. 1 the foreign gene cassettes are flanlked by 5.7 and 1.3 kbp of tobacco plastid DNA to mediate gene targeting by homologous recombination within the plastid. The plasmids are constructed from pTB27 containing tobacco plastid DNA (Sugiura et al., 1986). The regulatory elements driving expression of foreign uidA, aadA and bar genes are described in FIGS. 2-3. The bar gene described in White et al.(1991) was modified at the N and C termini and the resulting sequence shown in FIG. 4. The aadA gene was taken from pUC-atpX-AAD (Goldschmidt-Clermont, 1991) and the uidA gene is as previously described (Jefferson et al., 1986). Directly repeated copies of the NtpsbA terminator/3′ processing DNA sequence are distinguished by numbering. The two copies of the promoter-ribosome binding site region of rrnHv are distinguished as copy A or B. The directions of transcription of foreign genes are indicated.

Plastid transformation using pUM70 introduces a foreign DNA insert of 3.8 kbp containing the uidA and aadA genes into the plastid genome. pUM71 introduces a 4.9 kbp foreign insert, containing uidA, aadA and bat genes, into plastid genomes by transformation.

The rrnHv promoter (SEQ ID. NO. 15) was made by annealing oligonucleotides having SEQ ID No 1 and SEQ ID NO 2 and filling the single stranded regions with Taq DNA polymerase and deoxynucleotides.

| | |
|---|---|
| RBC-FL | |
| 5′AATAATCTGAAGCGCTTGGATACGTTGTAGGG-3′ | SEQ ID NO.1 |
| RBC-RL | |
| 5′CCCCCCATGGATGCCATAAGTCCCTCCCTACAAC-3′ | SEQ ID NO.2 |

The resulting fragment was used with primer HVRRNF (SEQ ID NO. 3) against pHvcP8 plasmid DNA (Day and Ellis, 1985) as template to amplify the 16SrRNA promoter linked to the ribosome binding site of the rbcL gene.

SEQ ID NO. 3.

5′CCCCCTCTAGACTCGAGTTTTTTCTATTTTGACTTAC-3′

The rrnBn promoter (SEQ ID NO. 16) was made by cloning the amplified 16SrRNA promoter region from purified Brassica napus chloroplast DNA with primers SAR5F (SEQ ID NO. 4) and XR3R (SEQ ID NO. 5)

| | |
|---|---|
| 5′CCCGCATGCCTTAGGTTTTCTAGTTGGATTTGC-3′ | SEQ ID NO.4 |
| 5′GGAGCCCGGGAGTTCGCTCCCAGAAAT-3′ | SEQ ID NO.5 |

This was ligated via a SmaI site to a synthetic ribosome binding site made by cloning annealed oligos RRT (SEQ ID NO. 6) and RRB (SEQ ID NO. 7) into MluI and NcoI digested vector DNA (pTrc99:aadA:NtpsbA).

| | |
|---|---|
| 5′CGCGTCCCGGGCGAATACGAAGCGCTTGGATAC-3′ | SEQ ID NO.6 |
| 5′CATGGATCCCTCCCTACAACTGTATCCAAGCGC-3′ | SEQ ID NO.7 |

The sequences of the synthesized rrnHv and rrnBn promoters are compared in FIG. 2. The sequences share approximately 78% base identity. Recombination between these promoters would not be expected to occur at a high frequency in transgenic plastids since they form an imperfect direct repeat in which the largest perfect duplication is only 17 bases long.

The NtPsbA terminator/3′ processing region was made using primers PSBA5F (SEQ ID NO. 8) and PSBA3R (SEQ ID NO. 9) against total Nicotiana tabacum DNA.

| | |
|---|---|
| 5′CCCAAGCTTCTGCAGGCCTAGTCTATAGGAGG-3′ | SEQ ID NO.8 |
| 5′GGGAAGCTTGGATCCTAAGGAATATAGCTCTTC-3′ | SEQ ID NO.9 |

The amplified product was cloned into the EcoRV site of pBluescript and the insert excised with PstI and HindIII or BamHI for cloning into the expression cassettes present in pUM70 and pUM71. Two copies of NtPsbA are present in pUM70 and three copies of NtPsbA are present in pUM71. The total length of the duplicated region involving NtPsbA is shown as SED ID. NO. 14 in FIG. 3 and includes linker sequences.

The 0.8 kbp NcoI-PstI fragment containing aadA coding sequences was obtained from pUC-atpX-AAD (Goldschmidt-Clermont, 1991). The 1.8 kbp NcoI-SmaI containing uidA coding sequence was taken from pJD330. The bar gene of Streptomyces hygroscopicus was obtained from plasmid pIJ4104 (White et al., 1990). The bar gene was modified by the introduction of an NcoI site at the start codon and the insertion of two TAA stop codons at the C-terminal end in place of its normal TGA stop codon (FIG. 4, SEQ ID NO. 17). The TAA stop codon is common in plastid genes and the insertion of tandem TAA stop codons ensures efficient chain termination. This was done using PCR primers BARF (SEQ ID NO. 10) and BARR (SEQ ID NO. 11).

| | |
|---|---|
| 5′CCCCCCCATGGGCCCAGAACGACGCCC-3′ | SEQ ID NO.10 |
| 5′TTATTAGATCTCGGTGACGGGCAG-3′ | SEQ ID NO.11 |

The resulting 570 bp coding sequence was cloned into the EcoRV site of pBluescript before insertion into the expression cassette present in pUM71 as a 570 bp NcoI-PstI restriction fragment. The expression cassettes containing foreign genes under the control of plastid regulatory regions were assembled in standard cloning vectors. For integration of the assembled foreign gene expression cassettes into the plastid genome they are cloned into a previously isolated fragment of chloroplast DNA. The plasmid pTB27 (Sugiura et al., 1986) was used to illustrate the procedure. To facilitate cloning a synthetic linker containing sites for ApaI and NotI was inserted into the AocI site of pTB27 present at position 59319 bp of the tobacco plastid genome (Shinozaki et al., 1986; DDBJ/EMBL/GenBank accession number z00044; Version 95 February 1999) to produce pTB27-link.

The synthetic linker was made by annealing oligonucleotides SEQ ID NO. 12 AND SEQ ID NO. 13.

| | |
|---|---|
| 5′TTAGGGCCCGGGAAAGCGGCCGC-3′ | SEQ ID NO.12 |
| 5′TAAGCCGCCGCTTTCCCGGGCCC-3′ | SEQ ID NO.13 |

Foreign gene cassettes were inserted between the NotI and ApaI sites of pTB27-link. The linker is located in the intergenic region between the rbcL and accD genes of tobacco plastid DNA. In the case of pUM71, the three foreign gene cassettes containing uidA, aadA and bar were excised with ClaI and NotI. The ClaI site was filled-in with deoxynucleotides and Klenow enzyme before ligation to ApaI (filled-in with deoxynucleotides and Klenow enzyme) and NotI digested pTB27-link.

The foreign genes in pUM70 -and pUM71 are flanked by 5.7 kbp and 1.3 kbp of tobacco plastid DNA to mediate integration into the plastid genome by homologous recombination (this integration event is illustrated in FIGS. 5 and 6).

### Example 2

### Plastid Transformation of Plants

Tobacco seeds (Nicotiana tabacum v. Wisconsin 38) were surface sterilised by immersion in 10% (W/V) sodium hypochlorite and gently shaken in jars at room temperature for 20 minutes. The seeds were then washed five times in sterile distilled water. Each wash lasted for 10 minutes. Seeds were germinated and propagated on agar solidified MS media (Murashige and Skoog, 1962) with 30 g/l sucrose.

A mixture of young and old leaves from a range of aseptic tobacco plants were cut and placed adaxial side downwards on solid RMOP medium (Svab et al., 1990). The leaves were positioned within a circle of 4 cm at the centre of a 9 cm petri-dish. A hole of approximately 0.5 cm was left free of leaves at the centre of this 4 cm circle. This arrangement of leaves, resembling a doughnut, maximises the efficiency of plastid transformation by localizing leaves to the spray areas where most plastid transformants are produced.

Approximately three milligrams of gold microprojectiles (1 μm) were first coated with 5 μg of plasmid DNA using spermidine and calcium chloride and finally resuspended in 65 μl of 100% ethanol. pUM70 and pUM71 were used as the coating plasmids. Five microlitres of plasmid coated gold suspension in ethanol were used per bombardment with the Bio-Rad PDS-1000 He particle delivery system. The petri-dish containing leaves was placed at shelf position 3 (approximately 9 cm from the rupture disk) and the leaves bombarded at 1,100 PSI at a vacuum of 27-28 mm Hg. Two spacer rings (5 mm) separated the stopping screen from the macrocarrier holder in the microcarrier launch system.

Bombarded leaves were allowed to recover for 40 to 48 hours before they were cut into small pieces of 3-5 mm in width and placed on RMOP medium containing 500 μg/ml spectinomycin and 500 μg/ml streptomycin. Plates were placed in stacks and incubated at 26° C. in a 12 hour light, 12 hour dark cycle with side illumination.

Primary resistant green shoots and green callus appeared after three to twenty weeks. In the case of pUM70 transformants shoots were cut into small pieces and placed on fresh RMOP solid medium containing spectinomycin and streptomycin. After regeneration of shoots on this medium they were re-cut for a second time and placed on fresh RMOP medium with spectinomycin and streptomycin. After a third cycle of regeneration on RMOP medium containing antibiotics shoots were transferred to magenta boxes containing solid MS medium supplemented with 100 μg/ml of spectinomycin. Once roots were produced plantlets were transferred to soil and allowed to recover for 5-10 days. The apical meristem and young leaves were sprayed weekly with a solution of spectinomycin (500 μg/ml), streptomycin (500 μg/ml) and 0.1 % (V/V) Tween 20 for 3-4 weeks.

In the case of pUM71, primary shoots and green cell lines resistant to spectinomycin and streptomycin were cut and transferred to solid RMOP medium containing 5 μg/ml of glufosinate-ammonium. After a second cycle of regeneration plants were transferred to magenta boxes containing MS medium supplemented with 1 μg/ml glufosinate-ammonium. Plantlets containing roots were transferred to soil and allowed to recover for 5-10 days. The apical meristem and young leaves of soil-growing plants were sprayed weekly with 0.1% V/V solution of Challenge TM (Hoescht “AgrEvo”), which contains glufosinate-ammonium, for a period of 3-5 weeks. The modified bar gene confers a high level of glufosinate tolerance to transplastomic plants (FIG. 13).

### Example 3

### Material Inheritance of Transplastomic Foreign Genes

On flowering, plastid transformants were crossed with flowers on non-transformed plants in reciprocal crosses. All progeny from crosses involving the plastid transformant as the maternal parent and non-transformed wildtype plants (WT) as the paternal parent were resistant to glufosinate-ammonium (FIG. 7). In contrast, all progeny from crosses involving plastid transformants as paternal (pollen-donor) parents were sensitive to glufosinate-ammonium. This maternal inheritance pattern of antibiotic or herbicide resistance is typical of a resistance gene integrated into plastid DNA.

### Example 4

### Excision of Antibiotic Resistance and Herbicide Resistance Genes From Transplastomic Plants

pUM71 contains three 418 bp directly repeated NtpsbA sequences (FIG. 3). These are numbered 1-3 in FIG. 5. It also contains two 174 bp directly repeated rrnHv sequences (FIG. 2) named A and B in FIG. 5 and an rrnBn sequence. Recombination between the rrnHv, and rrnBn promoter sequences would not be expected to occur at high frequency given their limited sequence identity (78% base identity, FIG. 2). Integration of the uidA, aadA and bar expression cassettes present in pUM71 replaces an 11.4 kbp HindIII plastid DNA fragment with two Hind III fragments of 6.9 and 9.5 kbp. This is shown in the scheme in FIG. 5. The 9.5 kbp Hind III fragment contains all three foreign genes (uidA, aadA, bar) linked to a junction fragment of tobacco plastid DNA. The integrated genes are located between the plastid rbcL and accD genes. This insertion event introduces a 4.9 kbp foreign DNA sequence into the tobacco plastid genome; the largest insertion described to date. Following the integration event, selection for the recombinant plastid genome of 161 kbp is maintained with glufosinate ammonium. This drives the plants to homoplasmy where all copies of the resident wild type plastid genome are replaced with recombinant plastid genomes containing the bar gene. In practice this is achieved by growing aseptic plants on media containing 5 μg/ml glufosinate ammonium and spraying soil grown plants with a 1:1000 dilution of the Challenge TM (AgrEvo) herbicide.

Once all the wild type plastid genomes have been replaced by recombinant plastid genomes the selection pressure is removed. This procedure selects for recombinant plastid genomes containing the bar gene. Such genomes will also contain the uidA and aadA genes unless they have been lost due to recombination event between the directly repeated 418 NtpsbA or 174 rrnHv regulatory sequences present in the foreign insert.

The recombination events leading to loss of the uidA, aadA and bar genes are shown in FIG. 5. Loss of the aadA and bar genes in Case 1 results from a recombination event between NtpsbA 1 and NtpsbA 3. In Case 2, loss of the uidA, and aadA gene results from a recombination event between rrnHv A and rrnHv B. In Case 3 recombination between NtpsbA 1 and NtpsbA 2 results in aadA loss. Loss of the bar gene in Case 4 results from recombination between NtpsbA 2 and NtpsbA 3. Lastly, recombination between rrnHv A and rrnBn would lead to uidA loss (Case 5). Recombination between rrnBn and rrnHv B would resemble Case 3 and is not shown.

Sixty transplastomic tobacco plants were generated from fifteen bombardments using pUM71. The sixty plants were derived from 48 independent transformation events. Fifty four of these plants were studied in detail. The intact cassette containing uidA, aadA and bar genes was present in 47 of the 54 plants studied. This results in the replacement of the 11.4 kbp wild-type plastid DNA HindIII band by bands of 9.5 kbp and 6.9 kbp in the plastid transformants. For examples see FIG. 8 and for an explanation see FIG. 5. The absence of a detectable 11.4 kbp band in the majority of transplastomic plants is consistent with replacement of the majority of wild type plastid genomes by recombinant plastid genomes containing foreign genes. A strong wild type 11.4 kbp plastid fragment was visible in three transplastomic plants (including 15A-11 in FIG. 8) indicating heteroplasmy of wild type and recombinant plastid genomes. The 9.5 kbp band contains all three foreign gene cassettes and hybridizes to DNA probes specific for the uidA, aadA and bar genes. All plants containing the intact 9.5 kbp band were glufosinate-ammonium resistant, spectinomycin resistant and contained readily detectable β-glucuronidase (GUS) activities. GUS is the product of the uidA gene. Hybridization of blots with a probe specific for nuclear ribosomal DNA (FIG. 8, bottom panel) demonstrated similar loadings of DNA per lane. Of the five plants that did not contain an intact foreign insert, four produced hybridization patterns indicating either mis-targeting or undesirable rearrangements and were not studied further.

Southern blot analysis was used to demonstrate the utility of the recombination events depicted in FIG. 5 to produce aadA-free and bar-free plastid genomes, which contain a gene of interest. Fifty-one transplastomic plants, from 48 independent clonal lines, obtained from 8 different bombardments were studied. Such a large sample size has allowed us to evaluate the frequency of the recombination events detailed in FIG. 5 with precision. Recombination events between NtpsbA1 and NtpsbA3 (Case 1 in FIG. 5) that excise aadA and the bar gene take place at high frequency to produce recombinant plastid genomes only containing the uidA gene. This is readily visualized as a 7.0 kbp Hind III band that hybridizes to the uidA gene in 35 of the 47 transformants that also contain the intact 4.9 kbp uidA-aadA-bar foreign insert. For an example see FIG. 8. In six of these 35 plants, the stochiometry of the 7.0 kbp HindIII band is similar (see 2 in FIG. 8) or higher than the 9.5 kbp band.

DNA samples from eleven pUM71 transplastomic plants produced minor 7.0 kbp HindIII bands which hybridized weakly to the uidA probe (for examples FIG. 8, lanes 15A-8, 15A-11). When the progeny of such plants (for example 13G in FIG. 9) were studied it was clear that production of recombinant plastid genomes only containing the uidA gene, due to aadA and bar gene loss, was a continual process. It accompanies the transmission of plastids through sexual crosses and mitotic cell divisions. For example, leaf samples from some of the T₁ progeny of parent plant 13G contain high levels of the marker-free plastid genome, revealed by a dark 7.0 kbp band (FIG. 9, bottom panel, lanes 3-6), whilst parent 13G (FIG. 9, lane 2) does not. The 133G parent did not contain WT plastid DNA and as expected, the 11.4 kbp WT band was not detectable in digests of DNA from progeny plants (FIG. 9, top panel, lanes 3-6). The stochastic processes of plastid DNA replication and segregation during cell division (cytoplasmic sorting) will also contribute to fluctuations in the relative levels of each genome type. The combined actions of gene excision and cytoplasmic sorting produce “marker-free” homoplasmic plastid transformants containing the uidA gene. Marker-free transplastomic seedlings bleach on media containing spectinomycin since they lack the aadA gene and resemble bleached WT seedlings (FIG. 10A). They represent approximately 24% (79/326) of T2 seedlings derived from the 13G-T1-2 parent, which contained high levels of the 7.0 kbp Hind III band diagnostic of “marker-free” plastid genomes (FIG. 9, bottom panel, lanes 5 and 6). White seedlings are not the result of mutations in plastid DNA since no white seedlings were observed when transgenic seeds were plated on media lacking spectinomycin (FIG. 10B). β-glucuronidase (GUS) activity was clearly observed in green T₂ seedlings from parent 13G-T1-2 but not in WT (FIG. 10C). Inhomogeneous staining is largely due to incomplete penetration of the GUS substrate (X-Gluc) into leaves. Bleached T₂ seedlings from parent 13G-T1-2 were transferred to medium lacking spectinomycin to allow recovery of plastid protein synthesis (FIG. 10D) and uidA gene expression. GUS activity in these seedlings was largely localised to green leaves (FIG. 10D) where restoration of plastid protein synthesis was complete. All tested spectinomycin-sensitive transplastomic plants were GUS positive and sensitive to glufosinate-ammonium.

The results of recombination events between NtpsbA 1 and NtpsbA 2 (FIG. 5, Case 3) and NtpsbA 2 and NtpsbA 3 (FIG. 5, Case 4) were not detected by Southern blot analysis. If these recombination events do take place our data suggest that the resulting products depicted in cases 3 and 4 in FIG. 5 are unstable (FIG. 11A). Further recombination between the duplicated NtpsbA regions in these products leads to a further gene loss whilst retaining uidA. The product of recombination in FIG. 5 case 4 has also been obtained by transforming tobacco plastid with pUM70. Southern blot analysis of seven independent pUM70 plastid transformants shows that the tandem uidA, aadA gene cassettes containing two NtpsbA repeats is relatively stable (FIG. 11B). None of these pUM70 plastid transformants lose the aadA gene a high frequency due to the absence of a predominant 7.0 kbp HindIII band (see scheme in FIG. 6). Therefore, our analyses of pUM71 plastid transformants suggest that three direct repeats activate a recombination pathway that lead to rapid loss of two of these repeats and the intervening DNA regions between them. Studies on pUM70 transformants suggest that the intermediates in the pUM71 recombination pathway containing two NtpsbA repeats are not intrinsically unstable.

Recombination events between rrnHv A and rrnHv B that lead to loss of uidA and aadA genes (FIG. 5, Case 2) take place at reduced frequency relative to recombination events between NtpsbA repeats. Recombinant plastid genomes only containing the bar gene are visualised as a 5.7 kbp band that hybridizes to the bar gene probe but not uidA or aadA probes (FIG. 5, Case 2). This 5.7 kbp band is a minor species in the majority of pUM71 transformants that contain the 9.5 kbp uidA-aadA-bar band. It is clearly visible in three pUM71 plastid transformants (for example see FIG. 8, transformant 130). This low excision frequency is sufficient to produce homoplasmic plastid transformants which lack the uidA and aadA genes but contain the bar gene (FIG. 8, transformants 14B and 14C). No hybridization was detectable in DNA from 14B and 14C using the aadA and uidA probes. Plants 14B and C were resistant to glufosinate-ammonium but sensitive to spectinomycin due to loss of the aadA gene. Enzyme assays for the product of the uidA gene, β-glucuronidase (GUS), showed no detectable activities in 14B and C. Recombination events between rrhHv A and rrnBn that result in loss of uidA were not detected (FIG. 5; Case 5).

### Example 5

### Irradiation of pUM70 Transplastomic Plants

pUM70 was transformed into tobacco plastids to produce stable transplastomic plants. pUM70-1 was shown to be homoplasmic for the recombinant 3.8 kb foreign insert containing the uidA and aadA genes by Southern blot analysis. Flowers from transformant pUM70-1 were crossed with pollen from untransformed WT tobacco plants. Germination of 500 seeds produced seedlings, all of which were spectinomycin-resistant. This maternal inheritance pattern is consistent with location of the foreign insert containing the aadA gene in the plastid genome. Separate batches of pUM70-1 transplastomic seeds were exposed to increasing doses of radiation from a cobalt source. The doses used were 50, 100, 150 and 200 krads. After surface sterilisation (30 min in 10% sodium hypochlorite, three washes in sterile distilled water) seeds were germinated on solid MS medium supplemented with 3% sucrose, 1 mg/L BAP and 0.1 mg/L NAA. After 3-4 weeks, plants were transferred to fresh medium. Within 3-4 weeks following transfer, a fraction of plants produced white sectors on leaves. The white and green shoots of these variegated plants were separated and propagated in parallel in vitro. In some cases, yellow shoots were observed which were unstable and produced wholly white or green shoots. A total of 25 lines were propagated as albino and green plants. All these lines were derived from seeds irradiated with 100-200 krads. Gamma irradiation would be expected to make double-stranded breaks in plastid DNA and induce DNA recombination and repair enzymes. Repair of double-stranded breaks can lead to deletions in plastid DNA that result in albinism. Therefore, albinism can provide an indicator of plastid genomes subject to increased recombination. These general recombination enzymes may be expected to act on the duplicated NtpsbA repeats flanking the aadA gene leading to its excision.

Green (G) and albino (A) plants derived from seven seeds were studied in detail by Southern blot analysis. The 8.3 kbp Hind III band results from integration of the intact foreign insert containing uidA and aadA genes into the plastid genome (FIG. 6) This 8.3 kbp HindIII is present in the majority of green and albino plants and hybridizes to the aadA and uidA gene probes (FIG. 12). Excision of the aadA gene produces a 7.0 kbp Hind III that only contains the uidA gene (FIG. 6). This 7.0 kbp band is visible in plants 2G, 7G and 7A which also contain an intact 8.3 kbp band. These plants are heteroplasmic and contain plastid genomes with an intact uidA-aadA insert and plastid genomes that have lost the aadA gene whilst retaining uidA. Excision of the aadA gene from the plastid genome in plant 5A has produced a “marker-free” transplastomic plant that contains the uidA gene. No aadA gene is detectable in DNA from plant SA by Southern blot hybridization (FIG. 12). Plant 5A contain the GUS enzyme but is spectinomycin sensitive since it lacks the aadA gene. Sensitivity is determined by the ability of plants to produce roots on spectinomycin containing media.

References

Day, A., and THN. Ellis. (1985) Deleted forms of plastid DNA in albino plants from cereal anther culture. Current Genet 9:671-678.

Goldschmidtclermont, M. (1991) Transgenic expression of aminoglycoside adenine transferase in the chloroplast: a selectable marker for site-directed transformation in Chlamydomonas. Nucleic Acids Research 19:4083-4089.

Jefferson, R. A., S. M. Burgess, and D. Hirsh. (1986) Beta glucuronidase from Escherichia coli as a gene fusion marker. Proc. Natl. Acad. Sci. USA. 83:8447-8451.

Murashige, T., and F. Skoog. (1962) A revised medium for rapid growth and bioassays with tobacco tissue cultures. Physiol Plant. 15:473-497.

Shinozaki, K., M. Ohme, M. Tanaka, T. Wakasugi, N. Hayashida, T. Matsubayashi, N. Zaita, J. Chunwongse, J. Obokata, K. Yamaguchishinozaki, C. Ohto, K. Torazawa, B. Y. Meng, M. Sugita, H. Deno, T. Kamogashira, K. Yamada, J. Kusuda, F. Takaiwa, A. Kato, N. Tohdoh, H. Shimada, and M. Sugiura. (1986) The complete nucleotide sequence of the tobacco chloroplast genome: its gene organization and expression. EMBO J 5:2043-2049.

Sugiura, M., K. Shinozaki, N. Zaita, M. Kusuda, and M. Kumano. (1986) Clone bank of the tobacco (Nicotiana tabacum) chloroplast genome as a set of overlapping restriction endonuclease fragments: mapping of 11 ribosomal protein genes. Plant Science 44:211-217.

Svab, Z., P. Hajdukiewicz, and P. Maliga. (1990) Stable transformation of plastids in higher plants. Proc Natl Acad Sci USA 87:8526-8530.

White, J., S. Y. P. Chang, and M. J. Bibb. (1990) A cassette containing the bar gene of Streptomyces hygroscopicus: a selectable marker for plant transformation. Nucl Acids Res 18:1062.

1. A method for producing a transgenic plant comprising a recombinant plastid genome containing an exogenous gene in the absence of a selectable marker gene introduced with the exogenous gene, the method comprising:
(a) stably transforming the plastid genome of a plant cell with nucleic acid comprising an exogenous gene, a selectable marker gene and at least two direct repeat sequences arranged to effect a recombination event within the transformed plastid genome to excise the selectable marker gene, whilst retaining the exogenous gene;
(b) selecting for transformed plants whose plastids comprise the selectable marker gene on a first selection medium; and
(c) growing the selected transformed plants in the absence of the first selection medium to promote excision of the selectable marker gene by recombination within the transformed plastid genome whilst retaining the exogenous gene.

2. A method according to claim 1 in which the plant cell is selected from a dicotyledonous plants such as a tobacco plant or other plants from the family Solanaceae, a plant from the family Brassicaceae, or monocotyledonous plants including plants from the family Gramineae such as a cereal or grass.

3. A method according to claim 1 in which the nucleic acid is stably transformed into the plastid genome by homologous recombination.

4. A method according to claim 1 in which the plastid genome is transformed with a nucleic acid construct comprising an expression cassette including an exogenous gene, a selectable marker gene and at least two direct repeat sequences.

5. A method according to claim 1 in which the plastid genome is co-transformed with two separate nucleic acid constructs, one comprising the selectable marker gene flanked by direct repeat sequences, the other comprising the exogenous gene.

6. A method according to claim 1 in which the exogenous gene is a gene for disease resistance, genes for pest resistance, genes for herbicide resistance, genes involved in specific biosynthetic pathways or genes involved in stress tolerance.

7. A method according to claim 6 in which the exogenous gene is the bar gene of Streptomyces hygroscopicus.

8. A method according to claim 1 in which the selectable marker is non-lethal.

9. A method according to claim 8 in which the selectable marker gene is the bacterial aadA gene.

10. A method according to claim 1 in which the direct repeat sequence comprises a nucleic acid sequences with little similarity to the plastid genome being transformed to reduce the opportunity of recombination between an inserted sequence and an endogenous sequence of the plastid occurring.

11. A method according to claim 1 in which the direct repeat sequence is at least 20 nucleotides in length.

12. A method according to claim 11 in which the direct repeat sequence is at least 50 nucleotides in length.

13. A method according to claim 12 in which the direct repeat sequence is at least 100 nucleotides in length.

14. A method according to claim 13 in which the direct repeat sequence is 174 nucleotides in length.

15. A method according to claim 14 in which the direct repeat sequence is 418 nucleotides in length.

16. A method according to claim 1 in which the direct repeat sequence is less than 10,000 nucleotides in length.

17. A method according to claim 1 in which the direct repeats flank the selectable marker gene.

18. A method according to claim 1 in which the nucleic acid to be introduced into the plastid genome comprises the exogenous gene and selectable marker gene with two direct repeats, one either side flanking the selectable marker gene.

19. A method according to claim 1 in which the nucleic acid to be introduced into a plastid genome comprises more than one exogenous gene and selectable marker gene with three direct repeats, two flanking the selectable marker gene and one flanking an exogenous gene.

20. A method according to claim 1 in which the nucleic acid to be introduced into a plastid genome comprises more than one exogenous gene and selectable marker gene with two direct repeats, one either side flanking the selectable marker gene.

21. A method according to claim 1 in which selection on the first selection medium is continued until homoplasmy.

22. A method according to claim 1 further comprising irradiating transformed plants grown on the first selection medium with gamma irradiation to promote excision of the selectable marker gene.

23. A method according to claim 1 for producing a transgenic tobacco plant comprising a recombinant plastid genome containing an exogenous uidA gene (encoding β glucuronidase) in the absence of the aadA gene introduced with the uidA gene.

24. A method according to claim 1 for producing a transgenic plant comlprisilng a recombinant plastid genome containing an exogenous gene in the absence of a first selectable marker gene introduced with the exogenous gene, the method comprising:
(a) stably transforming the plastid genome of a plant cell with nucleic acid comprising an exogenous gene, a first selectable marker gene and a second selectable marker gene and at least two direct repeat sequences arranged to effect a recombination event within the transformed plastid genome to excise the first selectable marker gene, whilst retaining the exogenous gene;
(b) selecting for transformed plants whose plastids comprise the first selectable marker gene on a first selection medium; and
(c) growing the selected transformed plants in a second selection medium to allow selection of plants containing the second selectable marker gene and to promote excision of the selectable marker gene by recombination within the transformed plastid genome whilst retaining the exogenous gene.

25. A method according to claim 24 in which the second selectable marker is the exogenous gene.

26. A method according to claim 25 in which the bar gene encoding phosphinothricin acetyltransferase is the second selectable marker.

27. A nucleic acid construct for transforming a plant plastid genome comprising at least two direct repeat sequences, an exogenous gene and a selectable marker gene wherein said construct is capable of transforming said plastid genome with subsequent loss of the selectable marker gene and retention of the exogenous gene in the plastid genome.

28. A nucleic acid construct according to claim 27 further comprising a second exogenous gene.

29. A nucleic acid construct according to claim 27 or 28 in which the direct repeat sequence is at least 20 nucleotides in length.

30. A nucleic acid construct according to claim 27 or 28 in which the direct repeat sequence is at least 50 nucleotides in length.

31. A nucleic acid construct according to claim 27 or 28 in which direct repeat sequence is at least 100 nucleotides in length.

32. A nucleic acid construct according to claim 27 or 28 in which the direct repeat sequence is 174 nucleotides in length.

33. A nucleic acid construct according to claim 27 or 28 in which the direct repeat sequence is 418 nucleotides in length.

34. A nucleic acid construct according to claim 27 or 28 in which the direct repeat sequence is less than 10,000 nucleotides in length.

35. A nucleic acid construct according to claim 27 or 28 in which the direct repeat sequence comprises a Ntpsb A sequence.

36. A nucleic acid construct according to claim 35 in which the Ntpsb A sequence is as shown as SEQ ID NO.14.

37. A nucleic acid construct according to claim 27 or 28 in which the direct repeat sequence comprises a rrnHv promoter sequence.

38. A nucleic acid construct according to claim 37 in which the rrnHv promoter sequence is as shown as SEQ ID NO.15.

39. A nucleic acid construct according to claim 27 or 28 in which the direct repeat sequence comprises a rrnBv promoter sequence.

40. A nucleic acid construct according to claim 39 in which the rrnBv promoter sequence is as shown as SEQ ID NO.16.

41. A nucleic acid construct according to claim 27 in which the exogenous gene, or gene is selected from genes for disease resistance, genes for pest resistance, genes for herbicide resistance, genes involved in specific biosynthetic pathways or genes involved in stress tolerance.

42. A nucleic acid construct according to claim 41 in which the exogenous gene is a uidA gene.

43. A nucleic acid construct according to claim 41 in which the exogenous gene is a bar gene.

44. A nucleic acid construct according claim 27 or 28 in which the selectable marker gene encodes a selectable marker that is non-lethal.

45. A nucleic acid construct according to claim 44 in which the selectable marker gene is a bacterial aadA gene.

46. A nucleic acid construct according to claim 28 in which the second exogenous gene is a selectable marker gene.

47. A nucleic acid construct according to claim 28 in which the second exogenous gene is a bar gene.

48. A nucleic acid construct according to claim 47 in which the bar gene is a modified bar gene comprising the sequence shown as SEQ ID NO. 17.

49. A nucleic acid construct according to claim 27 or 28 in which the direct repeat sequences flank the selectable marker.

50. A nucleic acid construct according to claim 28 in which there are at least three direct repeat sequences, two flanking the selectable marker gene and one flanking one of the exogenous genes.

51. A nucleic acid plasmid for transforming a plant plastid genome comprising a nucleic acid construct according to any one of claims 28 to 50.

52. Plasmid pUM71 comprising the bar gene, the uidA gene, the aadA gene, three copies of a directly repeated sequence of NtpsbA, two copies of a directly repeated sequence of rrnHv and one copy of rrnBv.

53. Plasmid pUM70 comprising the uidA gene, the aadA gene and two copies of a directly repeated sequence of NtpsbA.

54. Use of a plasmid according to any one of claims 51 to 53 to transform a plant plastid genome.

55. Use of a plasmid according to any one of claims 51 to 53 to transform a plant plastid genome according to the method of claim 1.

56. Transgenic plant cells or tissues comprising plastids transformed with a direct repeat and an exogenous gene or genes derived from the nucleic acid defined by claim 27 or 28.

57. Transgenic plant cells or tissues according to claim 56 produced according to the method of claim 1.

58. A transgenic plant comprising plastids transformed with a direct repeat and an exogenous gene or genes derived from the nucleic acid defined by claim 27 or 28.

59. A transgenic plant according to claim 58 comprising an exogenous bar gene.

60. A transgenic plant according to claim 59 in which the bar gene is provided using plasmid pUM71.

61. Use of the method according to claim 1 to produce glufosinate-ammonium resistant plants lacking a nucleus-located bar gene.
